(19) United States
(12) Patent Application Publication
(10) Pub. No.: US 2024/0139195 A1
(43) Pub. Date: May 2, 2024

(54) METHODS OF TREATING CHRONIC ACTIVE ANTIBODY-MEDIATED REJECTION USING BTK INHIBITORS

(71) Applicant: BeiGene Switzerland GmbH, Basel (CH)

(72) Inventor: Jiajun ZHOU, Changzhou, Jiangsu (CN)

(21) Appl. No.: 18/263,448

(22) PCT Filed: Jan. 29, 2022
(86) PCT No.: PCT/CN2022/074868

§ 371 Date: Jul. 28, 2023

(30) Foreign Application Priority Data
Jan. 30, 2021 (WO) PCTCN2021074565

(51) Int. Cl. A61K 31/519; A61K 45/06; A61P 37/06

(52) U.S. Cl. CPC A61K 31/519; A61K 45/06; A61P 37/06

(57) ABSTRACT

The present disclosure provides methods for treating chronic active antibody-mediated rejection (CAMR), in a subject, comprising administering to the subject a therapeutically effective amount of a BTK inhibitor, particularly (S)-7-(1-acryloylpiperidin-4-yl)-2-(4-phenoxyphenyl)-4,5,6,7-tetrahydropyrazolo-[1,5-a] pyrimidine-3-carboxamide or a pharmaceutically acceptable salt thereof.

### FIELD OF THE DISCLOSURE

Disclosed herein is a method for treating or preventing chronic active antibody-mediated rejection (CAMR) in a subject having an organ transplant, comprising administering to the subject a therapeutically effective amount of a BTK inhibitor, particularly (S)-7-(1-acryloylpiperidin-4-yl)-2-(4-phenoxyphenyl)-4,5,6,7-tetrahydropyrazolo-[1,5-a]pyrimidine-3-carboxamide or a pharmaceutically acceptable salt thereof.

### BACKGROUND OF THE DISCLOSURE

Antibody-mediated rejection (AMR) including chronic active antibody-mediated rejection (Chronic active AMR, CAMR), the major cause of chronic allogeneic rejection such as that in renal transplantation and lung transplantation, is a bottleneck of protection for graft function and improvement of recipient long-term survival rate. Despite development of some consensus diagnostic criteria, treatment protocols have limited data and long-term outcomes remain poor.

Renal transplantation is the most therapeutic approach for end-stage renal disease. Along with the development of immunosuppressive agents, T cell-mediated acute rejection after kidney transplantation has been effectively prevented. However, due to chronic rejection, the survival rate of allografts and allogeneic recipients is still unsatisfactory. The major cause of chronic rejection is the alloantibody mediated humoral immunity. Currently, the diagnosis of AMR has become more common and is a major cause of kidney graft loss, and there are no approved therapies and treatment guidelines are based on low-level evidence. CAMR, mainly mediated by humoral immunity, is a major cause of allograft failure. Treatments for CAMR include intravenous human immunoglobulin, plasmapheresis/immunoadsorption (PE/IA), rituximab and bortezomib. A number of retrospective studies have confirmed that the efficacy of intravenous immunoglobulin (IVIG) in the treatment of antibody-meditated rejection (ABMR) is uncertain [Ius F; et al. American journal of transplantation: official journal of the American Society of Transplantation and the American Society of Transplant Surgeons. 2018; 18: 2295-304; Matignon M, et al. PloS one. 2017; 12: e0178572.]. PE/IA is not an etiological treatment. Current studies of rituximab for the treatment of CAMR are only case reports. The treatment regimens used vary, and their effectiveness remains to be confirmed by randomized controlled trials [Pineiro G J, et. al. BMC nephrology. 2018; 19: 261; Parajuli S, et al. 2017; 3: e227; Oblak T; et al. 2017; 88: 91-6.]. Bortezomib, a highly selective proteasome inhibitor, has been reported to be able to prevent the production of alloantibodies to treat CAMR. Unfortunately, due to extensive adverse side effects of bortezomib, the application of bortezomib is still severely limited in the clinical treatment [De Sousa-Amorim E, et al. Nephrology (Carlton, Vic). 2016; 21: 700-4; Eskandary F, et al. Journal of the American Society of Nephrology: JASN. 2018; 29: 591-605.]. Therefore, identification of essential targets for designing rational strategies to treat patients with AMR including CAMR are of great need, and an effective approach treating CAMR and targeting the humoral allogeneic response in kidney transplantation are urgent requirements.

### SUMMARY OF THE DISCLOSURE

In an aspect, disclosed herein is a method for treating or preventing antibody-mediated rejection (AMR) in a subject having an organ transplant, comprising administering to the subject a therapeutically effective amount of a BTK inhibitor, or a pharmaceutically acceptable salt thereof.

In an aspect, the subject has undergone an organ transplant and exhibits symptoms of AMR of the transplanted organ.

In an aspect, the organ is one or more of heart, liver, lungs, pancreas or intestines.

In an aspect, the organ is the kidneys.

In an aspect, the antibody-mediated rejection comprises post-transplant AMR, chronic active ABMR (CAMR).

In an aspect, the antibody-mediated rejection is chronic active antibody-mediated rejection (CAMR). In some embodiments of the present disclosure, AMR or CAMR is related to chronic allogeneic rejection, such as that in allograft selected from renal transplantation and lung transplantation. In some embodiments of the present disclosure, the allograft is primary transplantation.

In an aspect, the organ is a kidney and the symptoms of CAMR comprise one or more of the following clinical and histological characteristics: (i) chronic transplant glomerulopathy (cg score>0) either with or without C4d deposition in peritubular capillaries and the presence of anti-HLA DSA determined by the local immunology laboratory; or (ii) stability of renal function defined as a decrease of eGFR<15% between the time of the diagnostic biopsy and the inclusion into the trial; and (iii) increased phosphorylation of Src and BTK. In some embodiments, the symptoms of AMR comprise all the above clinical and histological characteristics.

In an aspect, the BTK inhibitor or a pharmaceutically acceptable salt thereof is administered in combination with a therapeutically effective amount of an immune-suppressant.

In an aspect, the BTK inhibitor or a pharmaceutically acceptable salt thereof is administered in combination with a therapeutically effective amount of an immune-suppressant. In some embodiments of the present disclosure, the immune-suppressant targets the T-cell-mediated pathway. In some embodiments, the immune-suppressant is selected from cyclosporine, tacrolimus, mycophenolate, or mTOR inhibitors.

In an aspect, the BTK inhibitor is (S)-7-(1-acryloylpiperidin-4-yl)-2-(4-phenoxyphenyl)-4,5,6,7-tetrahydropyrazolo-[1,5-a]pyrimidine-3-carboxamide (Compound 1), ibrutinib, acalabrutinib or orelabrutinib, or a pharmaceutically acceptable salt thereof.

The present disclosure describes a BTK inhibitor, particularly (S)-7-(1-acryloylpiperidin-4-yl)-2-(4-phenoxyphenyl)-4,5,6,7-tetra-hydropyrazolo[1,5-a]pyrimidine-3-carboxamide (Compound 1) or a pharmaceutically acceptable salt thereof, showed sensitive response in some subjects with CAMR, and BTK could be a new target for the treatment of CAMR. BTK inhibitors, particularly Compound 1 could be used as a desensitization agent for chronic allogeneic rejection such as that in renal transplantation and lung transplantation kidney transplantation.

In the present disclosure, the inventor discovered that the phosphorylation of Src and BTK are remarkably increased in patients with chronic rejection, and Compound 1 showed the effects, including, alleviating CAMR; suppressing the elevation of B cells and plasma cells after kidney transplantation; preventing the infiltration of inflammatory cells in allogeneic kidneys, the activation of B cells via preventing the phosphorylation of BTK; reducing the secretion of proinflammatory cytokines and increased the secretion of anti-inflammatory cytokines, and protecting the allograft function, e.g., renal function, and improved the long-term survival rate of allogenic recipients.

### BRIEF DESCRIPTION OF THE DRAWINGS

FIG. 1: The phosphorylation of BTK was remarkably increased in patients with chronic rejection. (1A-1C) Patients in CR group showed significant severity of renal interstitial fibrosis, tubular atrophy and fibrous intimal thickening than Stable group according to the outcomes of HE staining and Masson staining. Bar=10 μm; Representative immunofluorescence staining of C4d (1C) and phosphorylated BTK (1E).

FIG. 2: Treatment with Compound 1 inhibited phosphorylation of BTK after kidney transplantation. After 4 weeks treatment of different doses of Compound 1, the expression and activity of phosphorylated BTK in allograft kidney was significantly inhibited by Compound 1 at dose of 2 mg/kg and 4 mg/kg. (2A) Representative immunofluorescence staining of phosphorylated BTK in allogeneic recipients treated with vehicle, and Compound 1 at different doses (0.2 mg/kg, 2 mg/kg, and 4 mg/kg). Bar=50 m. (2C) Equal amounts of protein from allograft kidney were analyzed by western blotting with antibodies against phosphorylated BTK, BTK, GAPDH after treatment with vehicle, and Compound 1 at different doses (0.2 mg/kg, 2 mg/kg, and 4 mg/kg) for 4 weeks. Data are relative abundance of protein or are expressed as individual spots per animal with means of each group (n=5) from 3 separate experiments. **P<0.01 versus allogeneic recipients treated with vehicle.

FIG. 3: Compound 1 alleviated the chronic active antibody-meditated rejection (CAMR) and reduced the IgG deposition in renal allografts. After the 8 weeks of transplantation, the allograft kidney developed into CAMR. The treatment of Compound 1 for 6 weeks from the second week significantly alleviated the CAMR. (3A) Donor specific antibody was produced by Lewis rats after 2 weeks after transplantation and reached the peak at 8 weeks after transplantation. Compound 1 significantly reduced the production of DSA at 8, 12, 16 weeks after transplantation. Data of MESF are expressed as the mean±SEM of each group (n=3) from 3 separate experiments. ^{a}P>0.05 versus syngeneic recipients treated with vehicle. ^{b}P>0.05 versus allogeneic recipients treated with Compound 1. ^{c}P>0.05 versus syngeneic recipients treated with Compound 1. ^{d}P<0.01 versus syngeneic recipients treated with vehicle. ^{e}P<0.01 versus allogeneic recipients treated with Compound 1. ^{f}P<0.01 versus syngeneic recipients treated with Compound 1. ^{g}P<0.05 versus syngeneic recipients treated with vehicle. ^{h}P<0.05 versus allogeneic recipients treated with Compound 1. ⁱP<0.05 versus syngeneic recipients treated with Compound 1. (3B) Representative HE, PAS, Masson staining of renal graft to show glomerular sclerosis, interstitial fibrosis, tubular atrophy and arteriosclerosis. Bar=10 m. (3D-3E) Representative immunofluorescence staining of C4d and IgG deposition. Bar=50 m. Data are representative images or are expressed as individual spots per animal with means of each group (n=5) from 3 separate experiments. Percentage of interstitial fibrosis, glomerular sclerosis, tubular atrophy, fibrous intimal thickening was presented as the mean±S.D. value of three independent experiments. **P<0.05 versus syngeneic recipients treated with vehicle. #P<0.05 versus allogeneic recipients treated with Compound 1. ^{##}P<0.05 versus allogeneic recipients treated with Compound 1. ⁺⁺P<0.05 versus syngeneic recipients treated with Compound 1. The black arrow determined the area defined by the internal elastic lamina.

FIG. 4: Compound 1 remarkably reduced the amount of B cells and plasma cells in rat peripheral blood. (4A-4D) 8 weeks after transplantation, T cells B cells and plasma cells significantly increased in rat peripheral blood. Allogeneic recipients were treated with Compound 1 (2 mg/kg) for 6 weeks starting at the second week after transplantation, which significantly inhibited increases of B cells and plasma cells, but only showed sight effect on T cells. Data are expressed as individual spots per animal with means of each group (n=5) from 3 separate experiments. *P<0.05 versus syngeneic recipients treated with vehicle. **P<0.01 versus syngeneic recipients treated with vehicle. ^{##}P<0.01 versus allogeneic recipients treated with Compound 1. ⁺P<0.05 versus syngeneic recipients treated with Compound 1.

FIG. 5: Compound 1 suppressed the inflammatory cells infiltration in allogeneic kidney. 12 weeks after kidney transplantation, T cells, B cells and macrophages were infiltrated in allogeneic kidney. Compound 1 remarkably reduced the infiltration of T cells, B cells and macrophages. (5A-5E) Representative immunofluorescence staining of T cells, B cells and macrophages. Bar=50 μm. Data are representative images or are expressed as individual spots per animal with means of each group (n=5) from 3 separate experiments. **P<0.01 versus syngeneic recipients treated with vehicle. ^{#}P<0.05 versus allogeneic recipients treated with Compound 1. ^{##}P<0.01 versus allogeneic recipients treated with Compound 1. ⁺P<0.05 versus syngeneic recipients treated with Compound 1. ⁺⁺P<0.01 versus syngeneic recipients treated with Compound 1.

FIG. 6: Compound 1 prevented the activation of B cells via inhibiting the phosphorylation of BTK. 8 weeks after kidney transplantation, the ratio of p-BTK⁺CD19⁺ cells were remarkably increased in the allogeneic kidney. The treatment of Compound 1 for 6 weeks remarkably inhibited the phosphorylation of BTK. (6A) Representative immunofluorescence staining of p-BTK and CD19. Bar=50 μm. (6C) Representative western blot of phosphorylated BTK, BTK, GAPDH. Data are representative images or are expressed as individual spots per animal with means of each group (n=5) from 3 separate assays. **P<0.01 versus syngeneic recipients treated with vehicle. ^{##}P<0.01 versus allogeneic recipients treated with Compound 1. ⁺P<0.05 versus syngeneic recipients treated with Compound 1. ⁺⁺P<0.01 versus syngeneic recipients treated with BGB-3111.

FIG. 7: Compound 1 protected renal function of the allogeneic recipients and prolongs survival of the recipients. The native right kidneys of recipient rats were resected 10 days after left kidney transplantation. (7A-B) The blood creatinine and urea nitrogen were continuously increased in allogeneic recipients treated with vehicle. Treatment of Compound 1 remarkably inhibited the increases of blood creatinine and urea nitrogen. (7C) The survival rate curve showed that Compound 1 significantly increased the long-term survival of allogeneic recipients. Data are expressed as the mean±SEM of each group from 3 separate experiments. *P<0.05 versus allogeneic recipients treated with vehicle. **P<0.01 versus allogeneic recipients treated with vehicle. Log-rank test showed the Compound 1 significantly increased the survival rate of allogenic recipients. P<0.05 versus allogeneic recipients treated with vehicle.

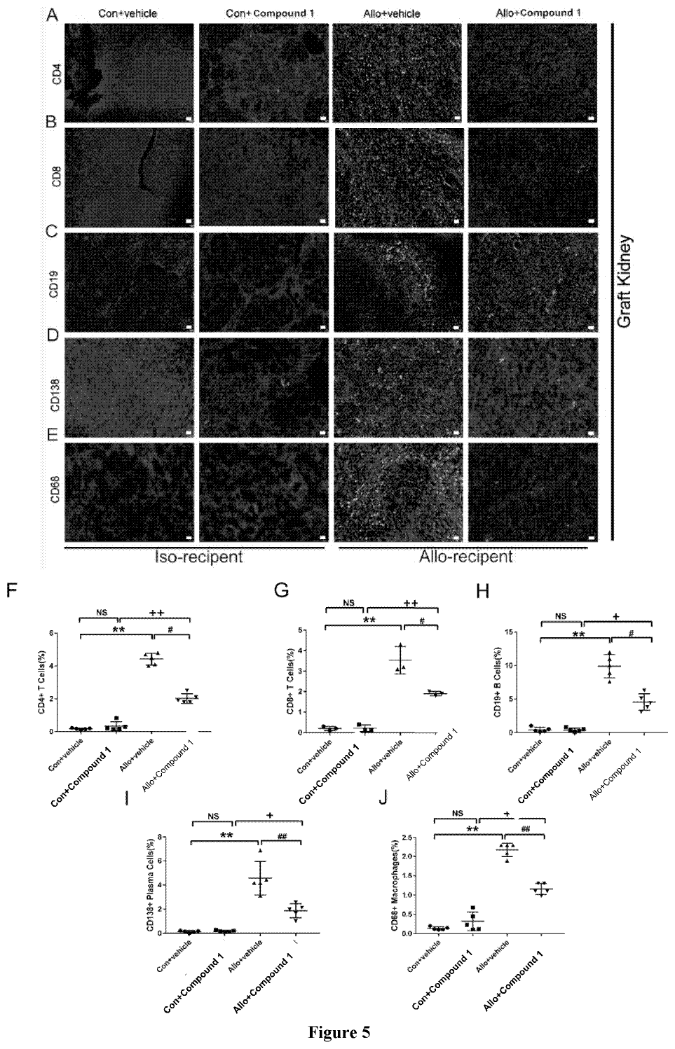

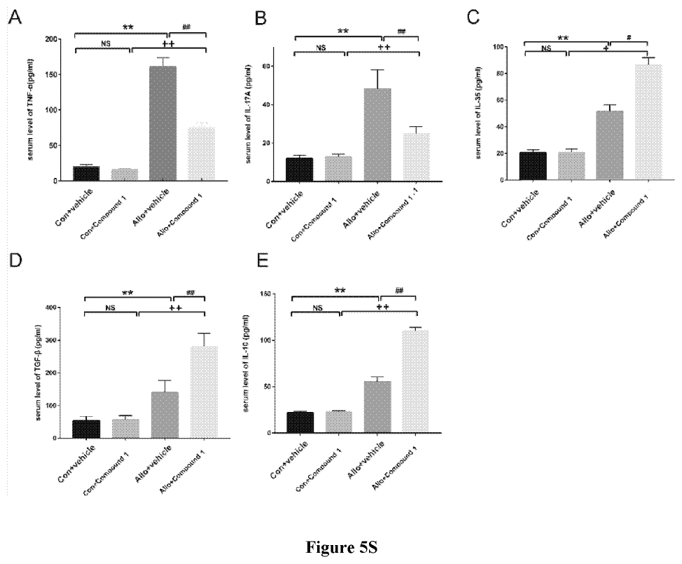

FIG. 5S: Compound 1 reduced the secretion of proinflammation cytokines TNF-α and IL-17A and increased the secretion of anti-inflammatory cytokines like IL-10, IL-35 and TGF-β. 12 weeks after transplantation, the cytokines, regardless of their anti-inflammatory or proinflammation effect remarkably increased in the allogeneic recipients. The treatment of Compound 1 could reduce the secretion of proinflammation cytokines TNF-α and IL-17A, but further increased the anti-inflammatory cytokines like IL-10, IL-35 and TGF-β. Data are presented as the mean±S.D. value of three independent experiments. **P<0.01 versus syngeneic recipients treated with vehicle. ^{#}P<0.05 versus allogeneic recipients treated with Compound 1. ^{##}P<0.01 versus allogeneic recipients treated with Compound 1. ⁺P<0.05 versus syngeneic recipients treated with Compound 1. ⁺⁺P<0.01 versus syngeneic recipients treated with Compound 1.

### DETAILED DESCRIPTION OF THE DISCLOSURE

### Definitions

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The terms “administration,” “administering,” “treating,” and “treatment” herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder, in particular, inhibiting and/or reducing the severity of antibody-mediated rejection of an organ transplant.

The term “therapeutically effective amount” as herein used, refers to the amount of a Bcl-2 inhibitor that, when administered to a subject for treating a disease, or at least one of the clinical symptoms of a disease or disorder, is sufficient to effect such treatment for the disease, disorder, or symptom. The “therapeutically effective amount” can vary with the agent, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition. In some embodiment of present disclosure, the subject is a human.

The present disclosure provides a method of treating antibody-mediated rejection (AMR) in a subject, comprising administering to the subject in need thereof Compound 1 or a pharmaceutically acceptable salt thereof.

The present disclosure also provides a method of treating chronic active antibody-mediated rejection (CAMR) in a subject, comprising administering to the subject in need thereof Compound 1 or a pharmaceutically acceptable salt thereof.

In some embodiments of the present disclosure, AMR or CAMR is related to chronic allogeneic rejection, such as that in allograft selected from renal transplantation and lung transplantation. In some embodiments of present disclosure, the allograft is a primary transplantation.

### Methods of Treatment

In one aspect, the present disclosure provides a method of treating AMR or CAMR in a subject.

In certain aspects, the method comprises administering to the subject in need thereof Compound 1 or a pharmaceutically acceptable salt thereof.

In some embodiments of present disclosure, AMR or CAMR is related to chronic allogeneic rejection, such as that in renal transplantation and lung transplantation kidney transplantation.

### BTK Inhibitor

BTK inhibitor is (S)-7-(1-acryloylpiperidin-4-yl)-2-(4-phenoxyphenyl)-4,5,6,7-tetrahydropyrazolo-[1,5-a]pyrimidine-3-carboxamide (Compound 1), ibrutinib, acalabrutinib or orelabrutinib, or a pharmaceutically acceptable salt thereof.

(S)-7-(1-acryloylpiperidin-4-yl)-2-(4-phenoxyphenyl)-4,5,6,7-tetra-hydropyrazolo[1,5-a]pyrimidine-3-carboxamide (Compound 1) was disclosed in the international publication No. WO2014/173289A.

Compound 1 can be administered by any suitable means, including oral, parenteral, intrapulmonary, and intranasal, and, if desired for local treatment, intralesional administration. Dosing can be by any suitable route. Various dosing schedules including but not limited to single or multiple administrations over various time-points, bolus administration, and pulse infusion are contemplated herein.

Compound 1 would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

### EXAMPLES

The present invention is further exemplified, but not limited to, by the following examples that illustrate the invention.

### Materials and Methods

Sample Collection

This was a study of adult patients who underwent primary renal transplantation. Recipients of multi-organ transplants or repeat renal transplantation were excluded. A total of 10 allograft segments, who had undergone kidney transplantation, were collected in CAMR group. Patients were classified into CR group with the following clinical and histological characteristics: chronic transplant glomerulopathy (cg score>0) either with or without C4d deposition in peritubular capillaries and the presence of anti-HLA DSA determined by the local immunology laboratory. Other inclusion criteria were age≥18 years, stability of renal function defined as a decrease of eGFR<15% between the time of the diagnostic biopsy and the inclusion into the trial. Moreover, 10 kidney samples from patients with stable allograft function were collected, who went through kidney transplantation. Exclusion criteria were eGFR<20 mL/min per 1.73 m2 at the time of inclusion, active neoplasia or history of neoplasia during the last 5 years except non-melanoma skin cancer, active bacterial, viral, or fungal infectious disease, and history of hypersensitivity reaction to any of the investigational products. Stable group was defined as serum creatinine (SCr) level was less than 120 μmol/L for at least 3 months following kidney transplantation. The baseline characteristics, clinical and histological characteristics of patients in the CR group, Stable group and control group are given in Table 1.

TABLE 1
Baseline, clinical and serologic characteristics
of the CAMR group and Stable group

| Variable | CAMR(n = 10) | Stable(n = 10) | P value |
|---|---|---|---|
| Age | 35.7 ± 3.01 | 31.86 ± 2.47 | NS |
| (years; mean ± SD) | | | |
| Gender | 5/5 | 5/5 | |
| (male/female) | | | |
| Transplant duration | 5.7 ± 1.2 | 4.5 ± 1.5 | NS |
| (years; range) | | | |
| Immunosuppression before biopsy | 5/1/4 | 4/2/4 | |
| (TAC + MMF + P/CsA + MMF + | | | |
| P/mTOR inhibitor + MMF + P) | | | |
| Biochemical parameters | | | |
| 1. Serum creatinine | 412 ± 198.1 | 92.1 ± 23.5 | 0.0001 |
| (μmol/L; mean ± SD) | | | |
| 2. BUN | 22.87 ± 8.98 | 8.12 ± 3.48 | 0.001 |
| (mmol/L; mean ± SD) | | | |
| Serologic characteristics | | | |
| 1. % PRA at transplant | 5.23 ± 5.17 | 6.83 ± 4.12 | NS |
| 2. HLA-A mismatches | 1.1 ± 0.5 | 1.4 ± 0.7 | NS |
| 3. HLA-B mismatches | 1.5 ± 0.6 | 1.7 ± 0.6 | NS |
| 4. HLA-DR mismatches | 1.4 ± 0.5 | 1.6 ± 0.7 | NS |
| 5. DSA class I/II/I + II | 1/7/2 | 0/1/1 | 0.01 |
| 6. MFI iDSA | 20780 ± 6780 | 1670 ± 110 | 0.001 |
| Proteinuria (g/day) | 1.7 ± 1.23 | 0.54 ± 0.37 | 0.001 |
| eGFR (mL/min per 1.73 m²) | 79 89 ± 5.89 | 22.12 ± 2.83 | 0.01 |

CAMR: chronic active antibody meditated rejection,
TAC + MMF + P, tacrolimus and mycohenolate mofetil and prednisone;
CsA + MMF + P, cyclosporin and mycophenolate mofetil and prednisone;
mTOR inhibitor + MMF + P, mammalian target of rapamycine inhibitor and mycophenolate and prednisone,
PRA: panel reaction antibody,
eGFR, estimated glomerular filtration rate,
DSA, donor specific antibodies,
MFI: mean fluorescence intensity,
iDSA: the DSA with the highest MFI level,
BUN: blood urea nitrogen,
NS: no significance

Animals

Inbred male F344 and Lewis rats (200 g to 250 g) were purchased from Charles River (Beijing, China). Animal handling procedures were conducted in compliance with guidelines for the Care and Use of Laboratory Animals published by the U.S. National Institutes of Health, and all animal experimental protocols were approved by Nanjing Medical University.

Kidney Transplantation

Left kidney transplantation was performed between male F344 and Lewis rats [Vogelbacher R, et al. Nephrology, dialysis, transplantation: official publication of the European Dialysis and Transplant Association—European Renal Association. 2010; 25: 3764-73.]. The average time of cold ischemia time and warm ischemia time was 25 and 40 minutes, respectively. In order to allow the production of alloantibodies, no immunosuppressive agents were used within the first 2 weeks after kidney transplantation.

Pharmaceutical Treatment and Tissue Harvest

From the second week after transplantation, Compound 1 at different doses (0.2 mg/kg, 2 mg/kg, and 4 mg/kg) in phosphate buffered saline was intravenously injected through tail vein twice a day. Recipients with phosphate buffered saline alone were considered as the vehicle control. At 4, 8, 12 and 16 weeks, organs were harvested and divided into two parts, which were fixed in paraffin or snap-frozen in N₂ and stored at −80° C. Peripheral blood mononuclear cells (PBMCs) were separated from the rat peripheral blood to be used in flow cytometry.

Donor Specific Alloantibody Analysis

Spleen lymphocytes of normal F344 rats were extracted using lymphocyte separation medium. Serum samples of allogeneic rats were incubated with spleen lymphocytes at room temperature for 30 minutes. Spleen lymphocytes were washed for three times and then incubated with a fluorescein isothiocyanate (FITC)-labeled anti-rat IgG antibody (BD Biosciences) at room temperature for 30 min. Mean fluorescence intensity (MFI) was determined in order to assess DSA levels by flow cytometry (Beckman DxFLEX, Beckman, Brea, CA) [Zhao D, et al. American journal of transplantation: official journal of the American Society of Transplantation and the American Society of Transplant Surgeons. 2018; 18: 1083-95; Liao T, et al. Frontiers in immunology. 2017; 8: 1334; Djamali A, et al. American journal of transplantation: official journal of the American Society of Transplantation and the American Society of Transplant Surgeons. 2014; 14: 255-71.]. A Quantum™ MESF Kit (Bangs Laboratories, San Francisco, CA) was used according to instructions of manufacturer of the kit. MFI was converted to MESF units, which is equivalent to adding internal parameters to the MFI and making the results more reliable, by using a data analysis program downloaded from www.bangslabs.com.

Flow Cytometry

PBMCs were doubly incubated with FITC-conjugated anti-CD45R (2.5 μg/10⁶ cells; eBioscience, Thermo Fisher Scientific, USA) and APC-conjugated anti-CD19 (2 μg/10⁶ cells; Bioss, China), FITC-conjugated anti-CD45R (2.5 μg/10⁶ cells; eBioscience, Thermo Fisher Scientific, USA) and Alexa Fluor 647 (conjugated anti-CD138 (0.1 μg/10⁶ cells; Abcam, USA), APC-conjugated anti-CD3 (3 μg/10⁶ cells; BioLegend, USA) and FITC-conjugated anti-CD4 (0.5 μg/10⁶ cells; BioLegend, USA) as well as APC-conjugated anti-CD3 (3 μg/10⁶ cells; BioLegend, USA) and Percp-eFluor710-conjugated anti-CD8 (0.3 μg/10⁶ cells; BioLegend, USA), respectively. The percentages of CD45R⁺CD19⁺B cells, CD45R⁻CD138⁺ plasma cells, and CD3⁺CD4⁺ and CD3⁺CD8⁺ T cells were determined by flow cytometry (BD Accuri C6, BD Biosciences).

Immunohistofluorescence

Paraffin-embedded sections (4 m) were incubated with primary antibodies against anti-phospho-BTK (1:100; Santa Cruz, USA), C4d (1:200, BioLegend, USA) CD3 (1:200, BioLegend, USA), CD4 (1:200, BioLegend, USA), CD8 (1:200, BioLegend, USA), CD19 (1:200, Bioss, China), CD138 (1:100, Abcam) and CD68 (1:100, Abcam) overnight at 4° C. in a humidified chamber. The appropriate isotype-matched IgG was used as the negative control. After washing, the bound antibodies were detected using FITC- or Cy3-conjugated secondary antibodies (1:200; Abcam), and images were acquired using a fluorescence microscope. Quantitative analysis of positive cells (T cells, B cells, plasma cell and macrophages) and IgG positive area was analyzed by using Image-Pro Plus (Media Cybernetics, Rockville, MD). Quantitative analysis of C4d positive PTC was performed according to outcomes of two independent pathologists blinded to the experimental design.

Western Blot

The experimental protocol of western blot was as described previously[Zhao C, et al. Gene. 2018; 642: 483-90.]. The primary antibodies are as followed: anti-phospho-BTK (1:1000; Santa Cruz), anti-BTK (1:1000; Santa Cruz, USA) and anti-GAPDH (1:1000; Cst, USA)

Histological Examination

Histological analysis was performed by using H&E, PAS and Masson staining. H&E, PAS and Masson trichome staining were performed as detailed elsewhere[Wang Z, et al. Journal of cellular and molecular medicine. 2017; 21: 2359-69.], which was used to evaluate the severity of CAMR. The diagnostic criteria of CAMR was according to Banff 2017 criteria. Characteristics of CAMR were defined as arterial intimal fibrosis, positive C4d staining in PTC and DSA. Measurement of arteriosclerosis caused by intimal fibrosis was performed for the areas surrounded by the luminal surface and internal elastic lamina of each vessel. The area determined by the internal elastic lamina subtraction of the internal elastic lamina to the luminal area was considered as the intimal area. Quantitative analysis the morphometric change of the kidney sections was performed according to results of two pathologists blinded to the experimental design independently.

Enzyme-Linked Immunosorbent Assay

The levels of rat serum TNF-α, TGF-β, IL-17A, IL-35, IL-10 were quantified by rat TNF-α ELISA kit (MUTISCIENCES; China), rat TGF-β ELISA kit (MUTISCIENCES; China), rat IL-35 ELISA kit (MUTISCIENCES; China), rat IL-17A ELISA kit (MUTISCIENCES; China) and rat IL-10 ELISA kit (MUTISCIENCES; China), respectively. The assays were performed as described in the manufacturer's instructions.

Renal Function Assessment

Concentrations of rat blood creatinine and urea nitrogen were tested by instructions of manufacturer of the kits (JianCheng, China).

Statistical Analysis

All data are presented as mean±S.D. Values are determined from three independent experiments. After demonstration of homogeneity of variance with Bartlett test, inter-group comparisons were made using one-way analysis of variance (ANOVA). Multiple means were compared using Tukey's test. The differences between two groups were determined by Student t-test. Values of P<0.05 were considered statistically significant. All the assays performed in triplicate.

### Example 1 the Phosphorylation of BTK is Remarkably Increased in the Allograft Kidney from Patients with CAMR

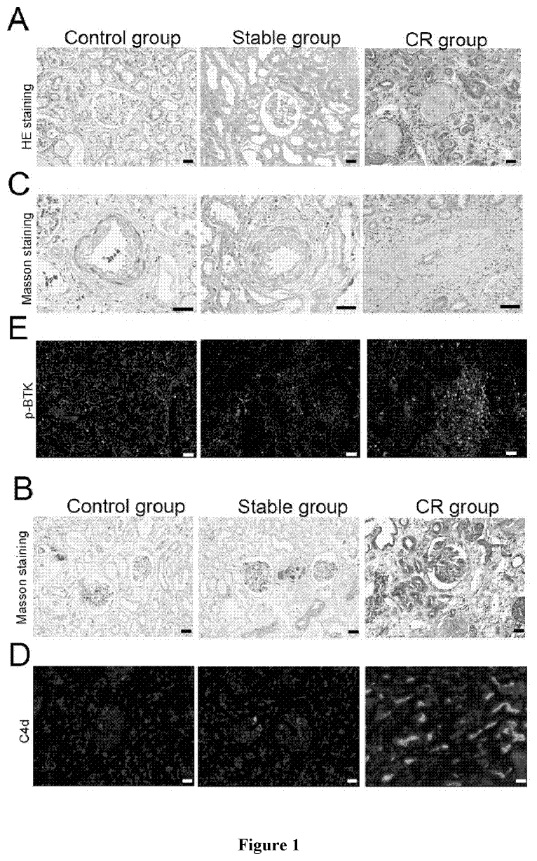

Ten patients with chronic active antibody meditated rejection (CAMR group) were included in this study. For comparison, same number of patients with stable renal allograft function (Stable group) were also recruited. The baseline, clinical and histological characteristics of patients and these healthy donors are given in Table 1. With the outcomes of HE and Masson staining (FIGS. 1A-1C), the inventors observed more severity of interstitial fibrosis, tubular atrophy and fibrous intimal thickening from allograft of CR group than Stable group. The number of C4d positive peritubular capillaries (PTC) was remarkably higher than Stable group (FIG. 1D). The phosphorylation of BTK was remarkably increased in the allograft kidney from patients with chronic rejection (FIG. 1E).

### Example 2 BTK Inhibitors Reduced the Phosphorylation of BTK after Rat Kidney Transplantation

Left kidney transplantation was performed between male F344 and Lewis rats. In order to determine the therapeutic dose of (S)-7-(1-acryloylpiperidin-4-yl)-2-(4-phenoxyphenyl)-4,5,6,7-tetrahydropyrazolo-[1,5-a]pyrimidine-3-carboxamide (Compound 1), Compound 1 was administrated to allogeneic Lewis rats. Compound 1 at different doses (0.2 mg/kg, 2 mg/kg or 4 mg/kg) was intravenously injected twice a day. After treatment with Compound 1 for 4 weeks, the graft kidneys were harvested and then the protein was extracted from the allograft. The phosphorylation of BTK was detected using immunofluorescence staining and western blot.

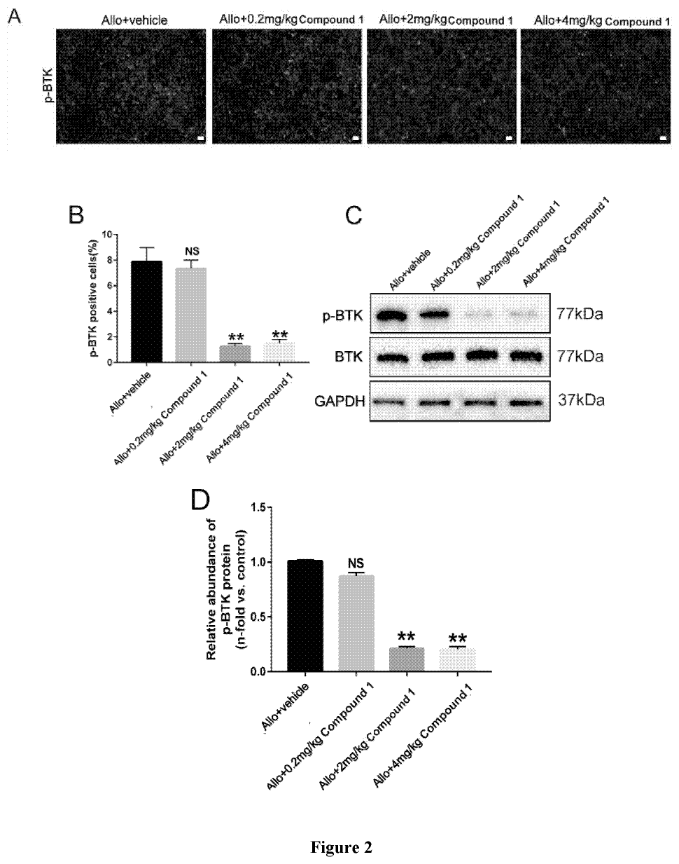

As shown in FIGS. 2A and 2B, the phosphorylation of BTK was remarkably inhibited by Compound 1, when the Lewis rats were treated with Compound 1 at doses of 2 mg/kg or 4 mg/kg. The outcomes of western blot also revealed that Compound 1 at dose of 2 mg/kg effectively inhibited the phosphorylation of BTK (FIGS. 2C-2D). Therefore, 2 mg/kg was determined as the treatment dose of Compound 1, to administrate in the following examples.

### Example 3 BTK Inhibitors Alleviated CAMR and Reduce IgG Deposition in Renal Allografts

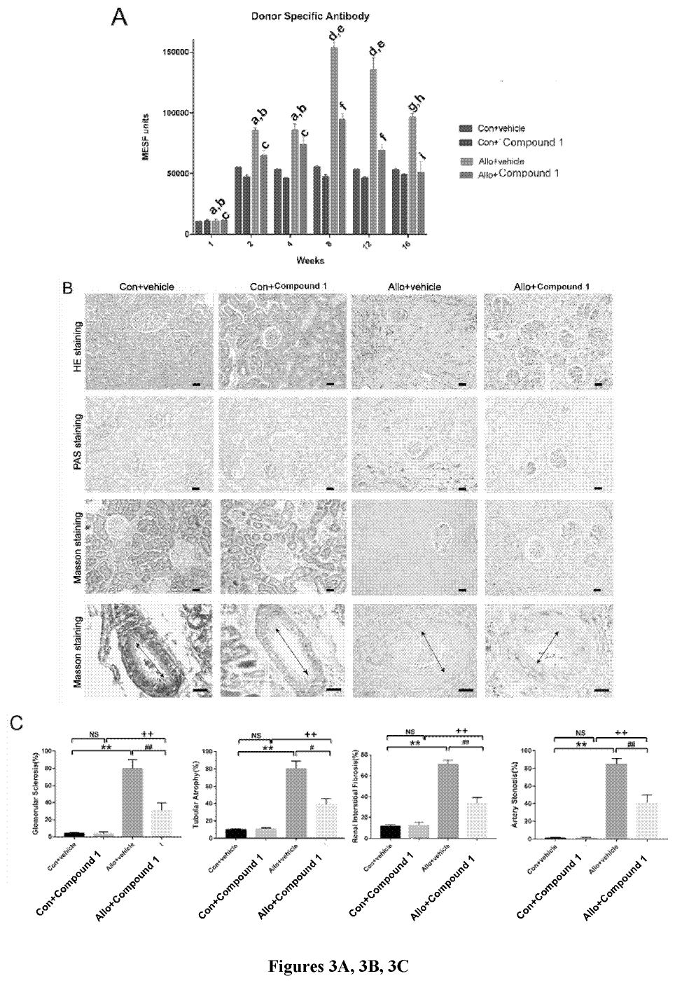

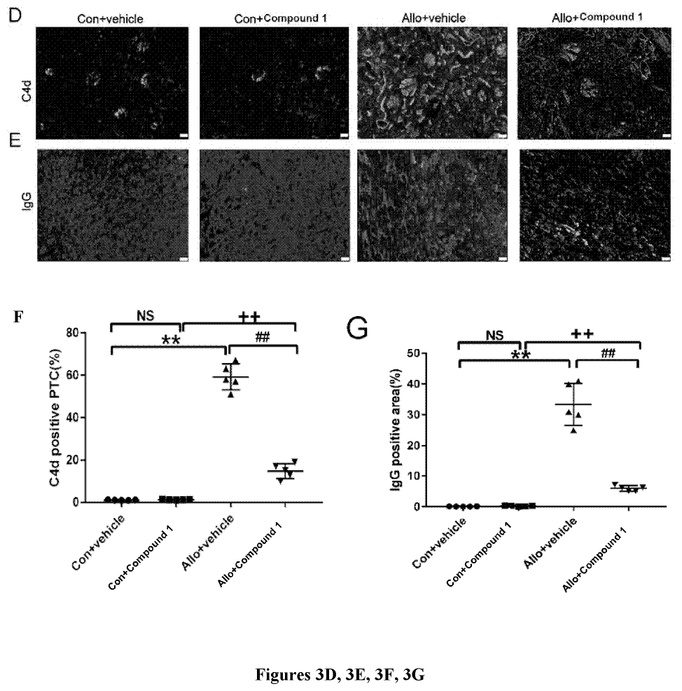

CAMR, which is defined by fibrous intimal thickening in arteries, deposition of C4d in PTC and an increase of DSA, was alleviated by the treatment of Compound 1. The levels of donor specific alloantibody (DSA) in allogenic and syngeneic recipients were tested at 1 week, 2 weeks, 4 weeks, 8 weeks, 12 weeks and 16 weeks after kidney transplantation (FIG. 3A).

At 2 weeks after kidney transplantation, the level of DSA started to increase. At 8, 12 and 16 weeks after kidney transplantation, compared with syngeneic recipients treated with vehicle, the level of DSA of allogenic recipients treated with vehicle was remarkably increased (FIG. 3A). In contrast, treatment with Compound 1 for the allogenic recipients starting at the second week after transplantation prevented further production of DSA, compared with allogeneic recipients treated with vehicle. The level of DSA was maintained at a relatively low level until the end of treatment at 16 weeks, compared with that of allogeneic recipients treated with vehicle (FIG. 3A).

The outcomes of hematoxylin and eosin (H&E) staining revealed that treatment with Compound 1 significantly ameliorated glomerular sclerosis, compared with that of allogeneic recipients with vehicle. The lesion area of tubular atrophy and interstitial fibrosis was over 70% on average for allograft kidneys according to the results of Periodic acid-Schiff (PAS) and Masson trichome staining. The ratio of interstitial artery stenosis reached 70% in allograft kidneys. After the treatment of Compound 1 for 10 weeks, the lesion areas of interstitial fibrosis, tubular atrophy and the ratio of interstitial artery stenosis were significantly ameliorated compared with that of allograft kidneys (FIGS. 3B-3C).

The deposition of C4d in peritubular capillaries (PTC) in allograft kidney was dramatically reduced after the treatment of Compound 1 (FIGS. 3D-3F). The positive area of IgG was almost 30% in allograft kidneys. After the treatment of Compound 1, the lesion area of IgG decreased to less than 10% (FIGS. 3E-3G).

### Example 4 BTK Inhibitors Suppressed the Elevation of B Cells and Plasma Cells after Kidney Transplantation

In order to investigate the immune state of syngenetic and allogenic rats, flow cytometry was used to test the amounts of T cells, B cells, and plasma cells. The outcome of flow cytometry revealed that CD3⁺CD4⁺ T helper cells, CD3⁺CD8⁺ cytotoxic T cells, CD19⁺CD45R⁺ B cells and CD138⁺CD45R⁻ plasma cells were significantly increased in peripheral blood.

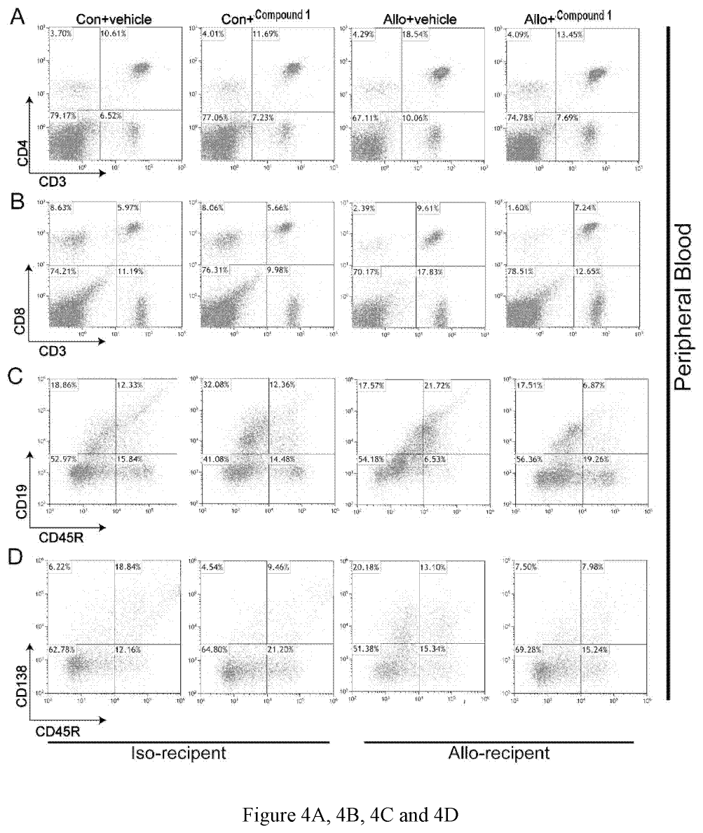

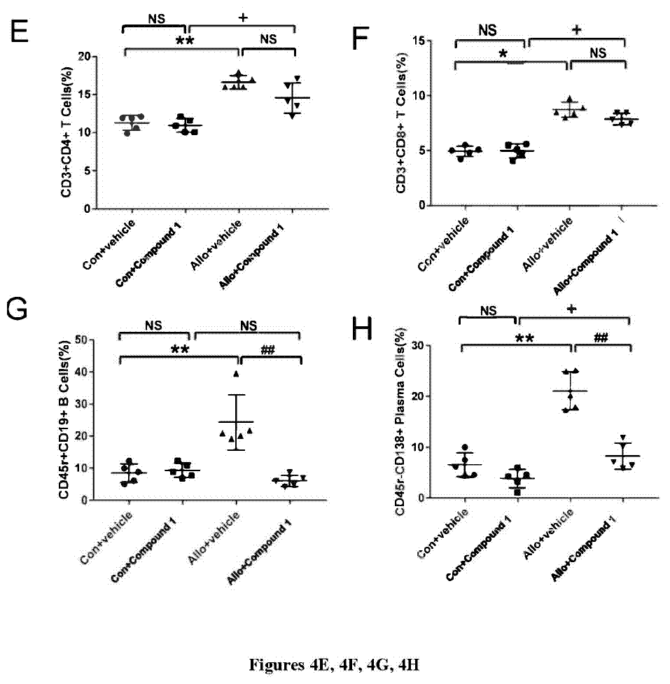

The treatment of Compound 1 significantly suppressed the elevation of CD19⁺CD45R⁺ B cells and plasma cells after 8 weeks of kidney transplantation. The ratio of CD19⁺CD45R⁺ B cells decreased from about 20% to 7%, while the ratio of CD138⁺CD45R⁻ plasma cells was suppressed from about 21% to 7%. However, Compound 1 showed slight effects on the T cells (FIGS. 4A-4D).

### Example 5 BTK Inhibitors Inhibited Inflammatory Cell Infiltration in Renal Allografts and Regulates the Secretion of Both Anti-Inflammatory and Proinflammatory Cytokines

Immunohistofluorescence was used to detect the influence of Compound 1 on inflammatory cells in graft kidneys. After 12 weeks of transplantation, allograft kidneys were significantly infiltrated with inflammatory cells including T cells, B cells, plasma cells and macrophages, compared with syngeneic recipients treated with vehicle.

Immunohistofluorescence revealed that Compound 1 significantly reduced the infiltration of T cells, B cells, plasma cells (FIGS. 5A-5D). A much more interesting finding would be that Compound 1 was able to decrease the infiltration of macrophages. The ratio of macrophages in allograft kidneys decreased from about 4% to 1.5% (FIG. 5E). It seemed that Compound 1 had an influence on macrophages.

At 12 weeks after kidney transplantation, serum isolated from rat peripheral blood was used to detect the concentration of inflammatory cytokines. Compared with syngeneic recipients treated with vehicle, serum levels of proinflammatory cytokines TNF-α and IL-17A significantly increased in allogeneic recipients treated with vehicle. However, Compound 1 treatment effectively decreased serum levels of TNF-α and IL-17A (FIGS. 5S A-5S B). Serum levels of anti-inflammatory cytokines, such as IL-10, IL-35 and TGF-β were still significantly increased in the allogeneic recipients treated by vehicle. However, Compound 1 treatment further increased the secretion of IL-10, IL-35 and TGF-β, compared with that of allogeneic recipients treated with vehicle (FIGS. 5S C-5S E). Compound 1 showed a good inhibitory effect on inflammation in allograft kidneys.

### Example 6 BTK Inhibitors Prevented the Activation of CD19*B Cells Via Reducing the Phosphorylation of BTK

In order to determine the effect of Compound 1 on B cells, immunohistofluorescence was used to detect the ratio of p-BTK⁺CD19⁺ B cells. CD19 positive B cells were represented by a red fluorescence while p-BTK positive cells were represented by a green fluorescence. CD19 and p-BTK double positive B cells were represented by a yellow fluorescence.

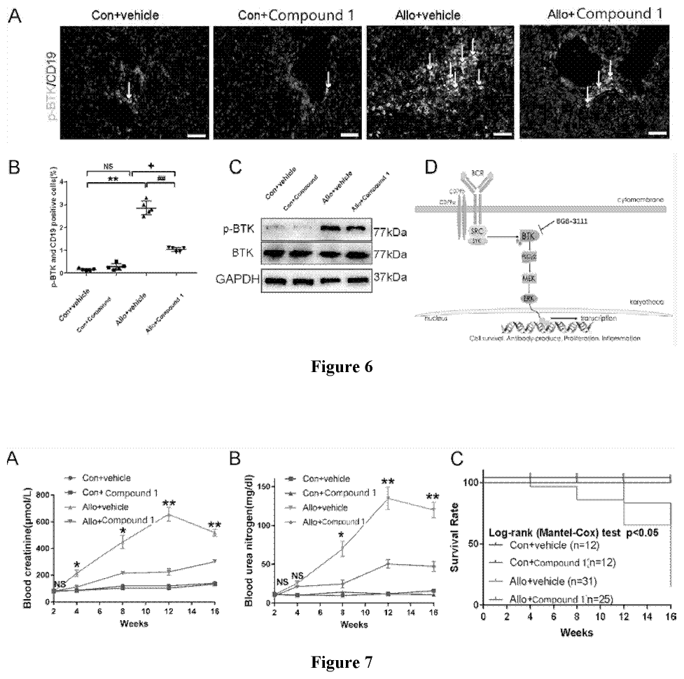

After Compound 1 treatment for 12 weeks, the ratio of p-BTK⁺CD19⁺ B cells significantly decreased (FIGS. 6A-6B). Western blot results also confirmed the finding that the phosphorylation of BTK was significantly decreased. (FIG. 6C). Taken together, the mechanism of Compound 1, which alleviates CAMR, may be that it blocks the B cell receptor signaling interactive pathway by inhibiting the phosphorylation of BTK (FIG. 6D).

### Example 7 BTK Inhibitors Protected Allograft Renal Function and Prolongs Survival of Allogenic Recipients

In order to examine the protective role of Compound 1 in graft kidney function and the long-term survival rate of allogenic recipients, nephrectomy of right kidney from Lewis rats was conducted at 10 days after kidney transplantation. After the resection of right kidney, the blood concentration of creatinine and urea nitrogen continued to deteriorate and reach the summit at 12 weeks after transplantation. However, because of Compound 1 treatment, there was on lasting deterioration of blood creatinine and urea nitrogen.

Blood creatinine and urea nitrogen remained at relatively low levels in allogeneic recipients treated with Compound 1 (FIGS. 7A-7B). Allogenic recipients treated with Compound 1 enjoyed a higher survival rate, compared with that of allogeneic recipients treated with vehicle (FIG. 7C).

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

1. A method for treating or preventing antibody-mediated rejection (AMR) in a subject having an organ transplant, comprising administering to the subject a therapeutically effective amount of a BTK inhibitor, or a pharmaceutically acceptable salt thereof.

2. The method of claim 1, wherein the subject has undergone an organ transplant and exhibits symptoms of AMR of the transplanted organ.

3. The method of claim 1, wherein the organ is one or more of heart, liver, lungs, pancreas or intestines.

4. The method of claim 1, wherein the organ is the kidneys.

5. The method of claim 1, wherein the antibody-mediated rejection comprises post-transplant AMR, chronic active ABMR (CAMR), and transplant glomerulopathy (TG).

6. The method of claim 1, wherein the antibody-mediated rejection is chronic active antibody-mediated rejection (CAMR).

7. The method of claim 1, wherein the organ is a kidney and the symptoms of CAMR comprise one or more of the following clinical and histological characteristics: (i) chronic transplant glomerulopathy (cg score>0) either with or without C4d deposition in peritubular capillaries and the presence of anti-HLA DSA determined by the local immunology laboratory; (ii) stability of renal function defined as a decrease of eGFR<15% between the time of the diagnostic biopsy and the inclusion into the trial; and (iii) increased phosphorylation of Src and BTK.

8. The method of claim 1, wherein the BTK inhibitor or a pharmaceutically acceptable salt thereof is administered in combination with a therapeutically effective amount of an immune-suppressant.

9. The method of claim 1, wherein the BTK inhibitor or a pharmaceutically acceptable salt thereof is administered in combination with a therapeutically effective amount of an immune-suppressant.

10. The method of claim 9, wherein the immune-suppressant targets the T-cell-mediated pathway.

11. The method of claim 10, wherein the immune-suppressant is selected from cyclosporine, tacrolimus, mycophenolate, or mTOR inhibitors.

12. The method of claim 1, wherein the BTK inhibitor is (S)-7-(1-acryloylpiperidin-4-yl)-2-(4-phenoxyphenyl)-4,5,6,7-tetrahydropyrazolo-[1,5-a]pyrimidine-3-carboxamide, ibrutinib, acalabrutinib or orelabrutinib, or a pharmaceutically acceptable salt thereof.

13. The method of claim 12, wherein the BTK inhibitor is (S)-7-(1-acryloylpiperidin-4-yl)-2-(4-phenoxyphenyl)-4,5,6,7-tetrahydropyrazolo-[1,5-a]pyrimidine-3-carboxamide, or a pharmaceutically acceptable salt thereof.
